(19) United States
(12) Patent Application Publication
(10) Pub. No.: US 2026/0179757 A1
(43) Pub. Date: Jun. 25, 2026

(54) PSEUDO-MASK-BASED ORGAN SEGMENTATION FOR MEDICAL IMAGES

(71) Applicants: Siemens Medical Solutions USA, Inc., Malvern, PA (US); Arizona Board of Regents on Behalf of the University of Arizona, Tucson, AZ (US)

(72) Inventors: Yanbo Zhang, Lawrenceville, NJ (US); Sabine Mollus, Jülich (DE); Stephan Kannengießer, Wuppertal (DE); Ali Bilgin, Tucson, AZ (US); Bernhard Geiger, Cranbury, NJ (US); Sasa Grbic, Plainsboro, NJ (US); Dorin Comaniciu, Princeton, NJ (US)

(21) Appl. No.: 18/986,823

(22) Filed: Dec. 19, 2024

(51) Int. Cl. G16H 30/40; G06T 7/12; G06T 7/30; G16H 30/20

(52) U.S. Cl. CPC G16H 30/40; G06T 7/12; G06T 7/30; G16H 30/20; G06T 2207/20081

(57) ABSTRACT

Systems and methods for generating pseudo-masks are provided. 1) a first and a second medical image of an anatomical structure of a patient and 2) a mask of the anatomical structure in the second medical image are received. The second medical image is aligned to the first medical image according to a deformation field. The mask of the anatomical structure in the second medical image is modified based on the deformation field to generate a pseudo-mask of the anatomical structure in the first medical image. The pseudo-mask of the anatomical structure in the first medical image is output.

### STATEMENT REGARDING FEDERALLY SPONSORED RESEARCH OR DEVELOPMENT

This invention is made with government support under 1U01EB031894-01A1 awarded by the National Institutes of Health. The government has certain rights in the invention.

### TECHNICAL FIELD

The present invention relates generally to AI/ML (artificial intelligence/machine learning) based medical imaging analysis, and in particular to pseudo-mask-based organ segmentation for medical images.

### BACKGROUND

MRI (magnetic resonance imaging) imaging provides a variety of pulse sequences, each with distinct imaging characteristics. Recently, machine-learning-based segmentation models have been proposed for segmenting anatomical structures from images of a specific MRI sequence. Such machine-learning-based segmentation models are typically trained on images of that specific MRI sequence. Currently, adapting such machine-learning-based segmentation models for segmenting anatomical structures from a different MRI sequence typically requires retraining or fine-tuning the machine-learning-based segmentation model on annotated images of that different MRI sequence. However, obtaining annotated images of the different MRI sequence is a time-consuming and costly process.

### BRIEF SUMMARY OF THE INVENTION

In accordance with one or more embodiments, systems and methods for generating pseudo-masks are provided. 1) a first and a second medical image of an anatomical structure of a patient and 2) a mask of the anatomical structure in the second medical image are received. The second medical image is aligned to the first medical image according to a deformation field. The mask of the anatomical structure in the second medical image is modified based on the deformation field to generate a pseudo-mask of the anatomical structure in the first medical image. The pseudo-mask of the anatomical structure in the first medical image is output.

In one embodiment, a machine-learning-based task network is trained for performing a medical imaging analysis task based on the first medical image and the pseudo-mask of the anatomical structure in the first medical image.

In one embodiment, the receiving, the aligning, the modifying, and the outputting are iteratively repeated for a training dataset to generate a plurality of the pseudo-masks. The training dataset comprises first training images of the anatomical structure, second training images of the anatomical structure, and training masks of the anatomical structure in the second training images. The iterative repeating is performed using one of the first training images as the first medical image, one of the second training images as the second medical image, and one of the training masks as the mask of the anatomical structure in the second medical image at each iteration. An initial training of the machine-learning-based task network for performing the medical imaging analysis task is performed based on the second training images and the plurality of pseudo-masks. Results of the medical imaging analysis task output by the initially trained machine-learning-based task network are evaluated by comparing the results with the plurality of pseudo-masks to determine whether each of the plurality of pseudo-masks represent high confidence data or low confidence data. The machine-learning-based task network is retrained for performing the medical imaging analysis task based on the plurality of pseudo-masks that represent high confidence data, without using the plurality of pseudo-masks that represent low confidence data.

In one embodiment, the plurality of pseudo-masks that represent low confidence data are adjusted.

In one embodiment, the medical imaging analysis task comprises segmentation of the anatomical structure from a medical image. In another embodiment, the medical imaging analysis task comprises detection of the anatomical structure from a medical image.

In one embodiment, the first medical image and the second medical image are in different domains. In one embodiment, the first medical image comprises a q-Dixon image and the second medical image comprises an e-Dixon image.

In one embodiment, a non-rigid and rigid registration between the second medical image and the first medical image is performed.

These and other advantages of the invention will be apparent to those of ordinary skill in the art by reference to the following detailed description and the accompanying drawings.

### BRIEF DESCRIPTION OF THE DRAWINGS

FIG. 1 shows a method for generating pseudo-masks, in accordance with one or more embodiments;

FIG. 2 shows a workflow for generating pseudo-masks, in accordance with one or more embodiments;

FIG. 3 shows a workflow for training a machine-learning-based segmentation model for segmenting an anatomical structure from a medical image, in accordance with one or more embodiments;

FIG. 4 shows a table comparing models trained using all pseudo-mask data and only high confidence pseudo-mask data generated in accordance with embodiments described herein;

FIG. 5 shows a table comparing models trained using all pseudo-masks and high confidence pseudo-masks generated in accordance with embodiments described herein with conventional segmentation models for segmentation of the liver;

FIG. 6 shows a table comparing models trained using all pseudo-masks and high confidence pseudo-masks generated in accordance with embodiments described herein with conventional segmentation models for segmentation of the spleen;

FIG. 7 shows an exemplary artificial neural network that may be used to implement one or more embodiments;

FIG. 8 shows a convolutional neural network that may be used to implement one or more embodiments;

FIG. 9 shows a data flow diagram of a generative adversarial network that may be used to implement one or more embodiments;

FIG. 10 shows a schematic structure of a recurrent machine learning model that may be used to implement one or more embodiments; and

FIG. 11 shows a high-level block diagram of a computer that may be used to implement one or more embodiments.

### DETAILED DESCRIPTION

The present invention generally relates to methods and systems for pseudo-mask-based organ segmentation for medical images. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for a novel approach to automatically generate pseudo-masks of anatomical structures across different MRI sequences. Such pseudo-masks may be utilized for training (including retraining or fine-tuning) machine-learning-based segmentation models for segmenting anatomical structures from medical images of the different MRI sequences, for example, where current machine-learning-based segmentation models do not provide support for such different MRI sequences. In one embodiment, a second medical image is aligned to a first medical image of an anatomical image according to a deformation field and a mask of the anatomical object in the second medical image is modified based on the deformation field to generate a pseudo-mask of the anatomical object in the first medical image. In another embodiment, a second medical image is aligned to a first medical image of the anatomical object and a mask of the anatomical object is extracted from the aligned second medical image, which represents a pseudo-mask of the anatomical object in the first medical image. Advantageously, embodiments described herein provide for automated pseudo-mask generation, significantly reducing or eliminating the need for manual annotation. Further, embodiments described herein allow machine-learning-based segmentation models to be adapted across different MRI sequences or modalities without the need for manual annotations, making them more versatile than conventional approaches that are limited to single-modality adaptation. In addition, embodiments described herein provide for a refinement step that iteratively improves the accuracy of pseudo-masks during the training process, addressing potential inaccuracies from the initial registration to mask generation.

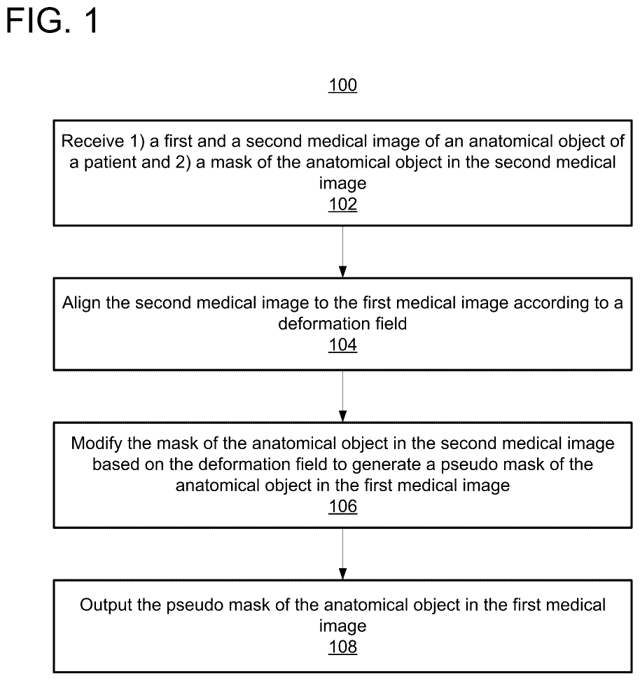

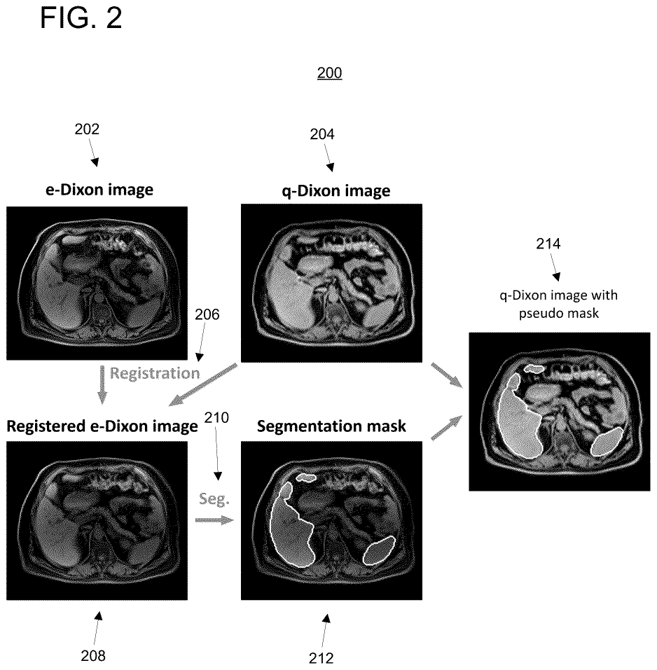

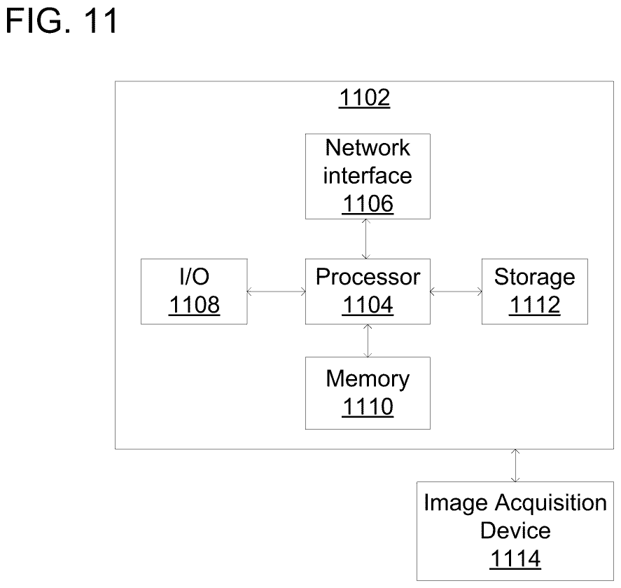

FIG. 1 shows a method 100 for generating pseudo-masks, in accordance with one or more embodiments. The steps and sub-steps of method 100 may be performed by one or more suitable computing devices, such as, e.g., computer 1102 of FIG. 11. FIG. 2 shows a workflow 200 for generating pseudo-masks, in accordance with one or more embodiments. FIG. 1 and FIG. 2 will be described together.

At step 102 of FIG. 1, a first and a second medical image of an anatomical structure of a patient and 2) a mask of the anatomical structure in the second medical image are received. In one example, the first medical image is q-Dixon (quantitative Dixon) image 204, the second medical image is e-Dixon (screening Dixon) image 202 and the mask of the anatomical structure is segmentation mask 212. A q-Dixon image refers to a (proton-density-weighted) multi-echo Dixon image while an e-Dixon image refers to a (T1-weighted) two-point Dixon image. q-Dixon and e-Dixon are variations of the Dixon method used in MRI for separating water and fat signals within the body of the patient. q-Dixon and e-Dixon enhance diagnostic capabilities in different clinical and research scenarios. In addition to original echo images, these may produce related images of water and fat, as well as parametric maps of, e.g., fat fraction (FF) and relaxation rate (R2*). As shown in workflow 200, e-Dixon image 202 and q-Dixon image 204 are water images.

The anatomical structure may comprise any suitable anatomical structure of interest of the patient. For example, the anatomical structure may comprise organs, bones, vessels, abnormalities (e.g., tumors), etc. In one embodiment, the first and second medical images are of different domains. As used herein, a domain of a medical image refers to the modality of the medical image as well as the protocol used for obtaining the medical image in that modality. The modality of the one or more input medical images may include, for example, MRI (magnetic resonance imaging), CT (computed tomography), US (ultrasound), x-ray, SPECT (single-photon emission computed tomography), PET (positron emission tomography), or any other medical imaging modality or combinations of medical imaging modalities. The protocol used for obtaining the medical image may include, for example, acquisition sequences or techniques for acquiring a medical image, such as, e.g., Dixon (e.g., e-Dixon or q-Dixon), T1-weighted, T2-weighted, proton density-weighted MRI images, contrast and non-contrast images, CT images captured with low kV (kilovoltage) and high kV, or low and high resolution medical images. Accordingly, the domains may be completely different medical imaging modalities or different image protocols within the same overall imaging modality. The first and second medical images may be 2D (two dimensional) images and/or 3D (three dimensional) volumes.

The mask of the anatomical structure in the second medical image is a segmentation mask that provides a pixel-wise (or voxel-wise) identification of the anatomical structure in the second medical image. Each pixel (or voxel) in the mask corresponds to a pixel (or voxel) in the second medical image and is assigned a value indicating whether the pixel depicts the anatomical structure in the second medical image.

The mask of the anatomical structure in the second medical image may be generated using any suitable (e.g., well-known) approach. In one embodiment, the mask of the anatomical structure in the second medical image may be generated using a machine-learning-based segmentation model trained to segment the anatomical structure for the domain of the second medical image. For example, as shown in workflow 200 of FIG. 2, segmentation mask 212 is generated from registered e-Dixon image 208. In another embodiment, the mask of the anatomical structure in the second medical image may be obtained by manually annotating the second medical image to identify the anatomical structure.

The first and second medical images and/or the mask of the anatomical structure in the second medical image may be received, for example, by directly receiving the first and second medical images from an image acquisition device (e.g., image acquisition device 1114 of FIG. 11) as the images are acquired, by loading the first and second medical images and/or the mask of the anatomical structure in the second medical image from a storage or memory of a computer system (e.g., storage 1112 or memory 1110 of computer 1102 of FIG. 11), or by receiving the first and second medical images and/or the mask of the anatomical structure in the second medical image from a remote computer system (e.g., computer 1102 of FIG. 11). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 104 of FIG. 1, the second medical image is aligned to the first medical image according to a deformation field. For example, as shown in workflow 200 of FIG. 2, e-Dixon image 202 is aligned to q-Dixon image 204 via registration 206 to provide for registered e-Dixon image 208.

The second medical image may be aligned to the first medical image using any suitable (e.g., well-known) registration technique to generate the deformation field. In one embodiment, the deformation field is a mathematical representation of a spatial transformation for aligning the second medical image (i.e., the moving image) to the first medical image (i.e., the fixed image). In one embodiment, the second medical image is aligned to the first medical image via rigid or non-rigid registration (deforming via translations and rotations).

At step 106 of FIG. 1, the mask of the anatomical structure in the second medical image is modified based on the deformation field to generate a pseudo-mask of the anatomical structure in the first medical image. In one example, as shown in workflow 200 of FIG. 2, segmentation mask 212 of e-Dixon image 202 is modified to generate a pseudo-mask of the anatomical structure of the q-Dixon image 204, which is represented in FIG. 2 as q-Dixon image with pseudo-mask 214.

At step 108 of FIG. 1, the pseudo-mask of the anatomical structure in the first medical image is output. For example, the pseudo-mask of the anatomical structure in the first medical image can be output by displaying the pseudo-mask on a display device of a computer system (e.g., I/O 1108 of computer 1102 of FIG. 11), storing the pseudo-mask on a memory or storage of a computer system (e.g., memory 1110 or storage 1112 of computer 1102 of FIG. 11), or by transmitting the pseudo-mask to a remote computer system (e.g., computer 1102 of FIG. 11).

In one embodiment, the first medical image and the pseudo-mask of the anatomical structure in the first medical image may be used for training a machine-learning-based task network for performing a medical imaging analysis task. The medical imaging analysis task may be segmentation of the anatomical structure. However, the medical imaging analysis task may comprise any other suitable medical imaging analysis task, such as, e.g., detection of the anatomical structure.

In one embodiment, the mask of the anatomical structure in the second medical image is not received at step 102 of FIG. 1. Method 100 proceeds to align the second medical image to the first medical image at step 104. Instead of performing step 106, a mask of the anatomical structure is extracted from the aligned second medical image. The mask of the anatomical structure in the aligned second medical image represents the pseudo-mask of the anatomical structure in the first medical image. The pseudo-mask of the anatomical structure in the first medical image is then output at step 108.

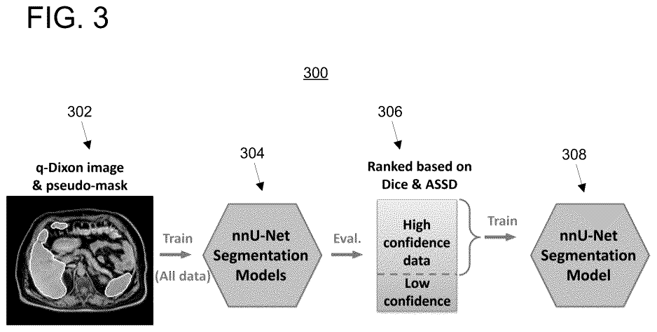

FIG. 3 shows a workflow 300 for training a machine-learning-based segmentation model for segmenting an anatomical structure from a medical image, in accordance with one or more embodiments. Training is performed using training dataset 302 comprising training medical images and pseudo-masks of the anatomical structure in the training medical images. In one embodiment, training dataset 302 is generated according to method 100 of FIG. 1 and workflow 200 of FIG. 2, wherein the training medical images are the first medical image and the pseudo-masks of the anatomical structure in the training medical images are the pseudo-mask of the anatomical structure in the first medical image.

Machine-learning-based segmentation model 304 is initially trained using training dataset 302. In one example, as shown in workflow 300 of FIG. 3, machine-learning-based segmentation model 304 is an nnU-Net (no-new-Net). However, machine-learning-based segmentation model 304 may be implemented according to any other suitable machine-learning-based architecture. The segmentation masks of the anatomical structure in the training medical images generated by the trained machine-learning-based segmentation model 304 are compared with the pseudo-masks. The segmentation masks generated by trained machine-learning-based segmentation model 304 are evaluated 306 based on the comparison to determine whether the pseudo-masks represent high confidence data or low confidence data. The evaluation 306 may be based on the mean Dice score and/or ASSD (average symmetric surface distance). The high confidence data may be considered the top N evaluated segmentation masks (where N may be a percentage (e.g., 66.6%) or a predefined positive integer), but may be determined using any other suitable approach. The machine-learning-based segmentation model 308 is then retrained using only the training medical images and pseudo-masks of the anatomical structure in the training medical images in training dataset 302 evaluated 306 as being high confidence data (and not the pseudo-masks evaluated 306 as being low confidence data).

In one embodiment, the segmentation masks generated by trained machine-learning-based segmentation model 304 that are evaluated 306 as being low confidence data are iteratively refined during the training process to progressively adjust the pseudo-masks to improve their accuracy and reliability.

Embodiments described herein were experimentally validated using two liver MRI datasets: Dataset A and Dataset B.

Dataset A (185 patients) includes e-Dixon and q-Dixon sequences per patient without direct organ mask annotations. The e-Dixon images have 60-96 slices per patient, with resolutions ranging from 0.9375 mm (millimeter) to 1.5972 mm and 3.0 mm slice thickness. The q-Dixon images have 60-96 slices, resolutions between 1.9375 mm and 2.875 mm, and 3.0 mm slice thickness. This dataset is used to develop models and there was no organ mask annotation in this dataset.

Dataset B (20 patients) includes only q-Dixon sequences, with expert annotations for liver and spleen masks. The images have 36 slices per case, resolutions from 2.0833 mm to 2.625 mm, and 5.0 mm slice thickness.

Pseudo-mask generation: In the pseudo-mask generation stage, non-rigid registration is applied to align the e-Dixon water image with the q-Dixon water image for each patient case. An organ segmentation model previously developed for e-Dixon water is then applied to the e-Dixon images to generate liver and spleen masks. These masks serve as pseudo-masks for the corresponding q-Dixon images, forming data pairs for subsequent model development.

Confident data selection and model training: To mitigate the impact of potential errors from both the e-Dixon segmentation and registration steps, only high-confidence data is used in the final model training phase. This process involves 3 steps. Firstly, initial 3D nnUNet v2 models were trained on all pseudo-mask data, employing a 5-fold cross-validation approach on Dataset A. During this phase, segmentation accuracy is assessed for each pseudo-mask case using Dice scores and ASSD metrics. Then, all cases are ranked by their Dice scores and ASSD metrics. The top-performing ⅔ of cases were selected for the final training phase, excluding the lower ⅓ of data which tends to contain noisier masks or less precise segmentation. The final nnUNet model is then trained on this subset of high-confidence data. By filtering out the lowest-performing masks, the noise introduced by pseudo-masks with lower accuracy is reduced, resulting in a more robust model.

Evaluation: The model was evaluated in two phases. First, models trained on all pseudo-mask data are compared against those trained on the high-confidence subset within Dataset A. Second, the final model trained on high-confidence data is benchmarked against TotalSegmentator and MRSegmentator segmentation models on Dataset B for liver and spleen segmentation, which provides a clear indication of the effectiveness of the embodiments described herein.

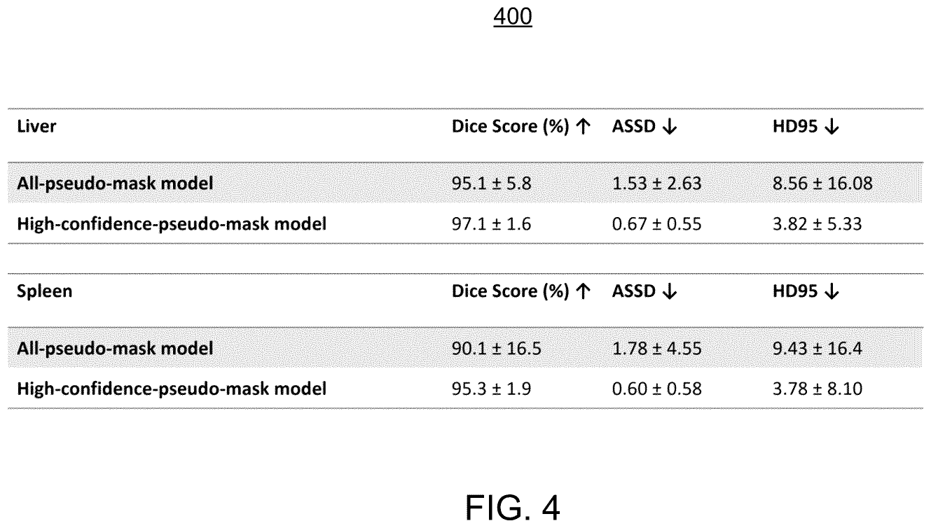

FIG. 4 shows a table 400 comparing models trained using all pseudo-mask data and only high confidence pseudo-mask data generated in accordance with embodiments described herein. The comparison is performed using Dice scores, ASSD, and HD95 (95th percentile of the Hausdorff distance). As shown in table 400, training the nnUNet model with high-confidence pseudo-masks significantly improves segmentation accuracy, with higher Dice scores and better ASSD values compared to training on all pseudo-mask data.

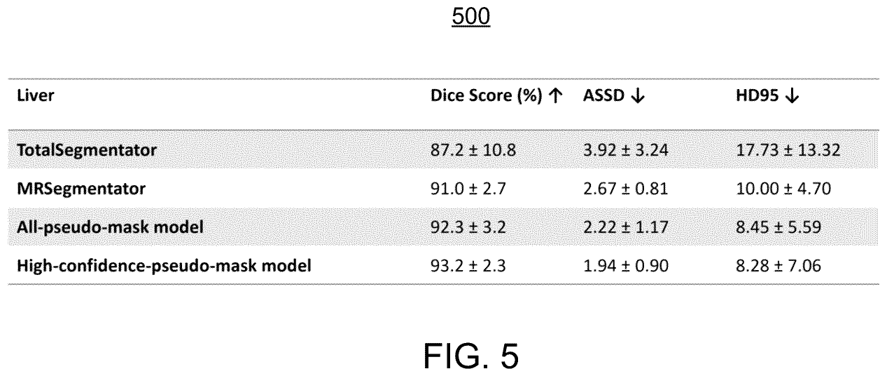

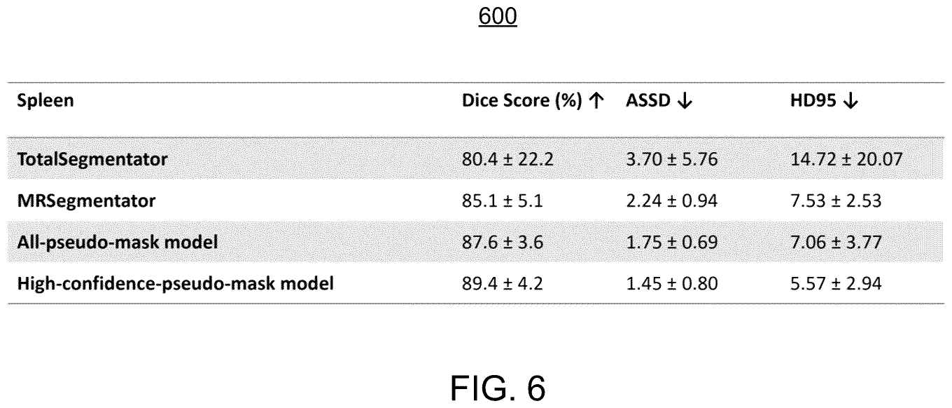

FIG. 5 shows a table 500 comparing models trained using all pseudo-masks and high confidence pseudo-masks generated in accordance with embodiments described herein with TotalSegmentator and MRSegmentator segmentation models for segmentation of the liver. FIG. 6 shows a table 600 comparing models trained using all pseudo-masks and high confidence pseudo-masks generated in accordance with embodiments described herein with TotalSegmentator and MRSegmentator segmentation models for segmentation of the spleen. As shown in tables 500 and 600, the models trained using all pseudo-masks and high confidence pseudo-masks generated in accordance with embodiments described herein outperform both TotalSegmentator and MRSegmentator segmentation models in liver and spleen segmentation.

The pseudo-mask segmentation method in accordance with embodiments described herein demonstrated superior performance on q-Dixon water MRI, surpassing conventional models. This annotation-free approach highlights the potential of pseudo-labels in medical image analysis, offering a practical solution to data scarcity.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, machine learning model utilized herein, including, e.g., the machine-learning-based model for generating the mask of the anatomical structure in the second medical image received at step 102 and the machine-learning-based model for performing step 104 of FIG. 1, the machine-learning-based model for performing registration 206 and segmentation 210 of FIG. 2, and the machine-learning-based segmentation models 304 and 308 of FIG. 3, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

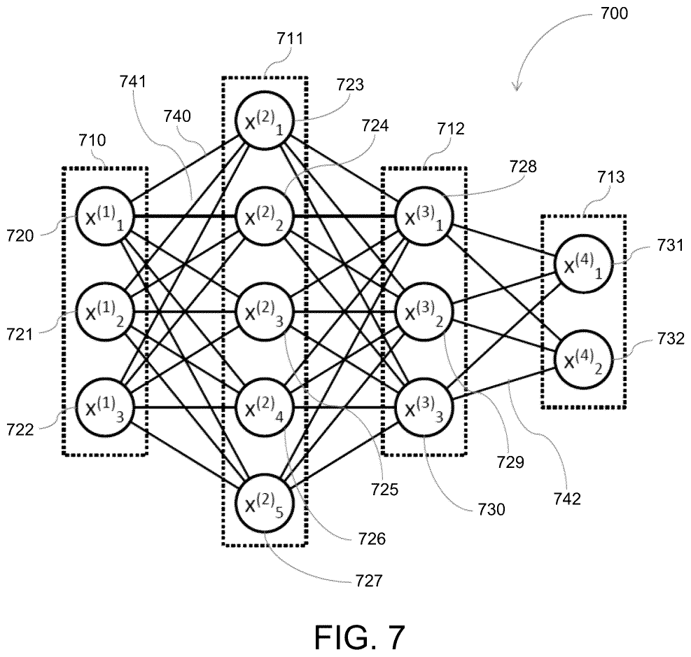

FIG. 7 shows an embodiment of an artificial neural network 700 that may be used to implement one or more machine learning models described herein. Alternative terms for “artificial neural network” are “neural network”, “artificial neural net” or “neural net”.

The artificial neural network 700 comprises nodes 720, . . . , 732 and edges 740, . . . , 742, wherein each edge 740, . . . , 742 is a directed connection from a first node 720, . . . , 732 to a second node 720, . . . , 732. In general, the first node 720, . . . , 732 and the second node 720, . . . , 732 are different nodes 720, . . . , 732, it is also possible that the first node 720, . . . , 732 and the second node 720, . . . , 732 are identical. For example, in FIG. 7 the edge 740 is a directed connection from the node 720 to the node 723, and the edge 742 is a directed connection from the node 730 to the node 732. An edge 740, . . . , 742 from a first node 720, . . . , 732 to a second node 720, . . . , 732 is also denoted as “ingoing edge” for the second node 720, . . . , 732 and as “outgoing edge” for the first node 720, . . . , 732.

In this embodiment, the nodes 720, . . . , 732 of the artificial neural network 700 can be arranged in layers 710, . . . , 713, wherein the layers can comprise an intrinsic order introduced by the edges 740, . . . , 742 between the nodes 720, . . . , 732. In particular, edges 740, . . . , 742 can exist only between neighboring layers of nodes. In the displayed embodiment, there is an input layer 710 comprising only nodes 720, . . . , 722 without an incoming edge, an output layer 713 comprising only nodes 731, 732 without outgoing edges, and hidden layers 711, 712 in-between the input layer 710 and the output layer 713. In general, the number of hidden layers 711, 712 can be chosen arbitrarily. The number of nodes 720, . . . , 722 within the input layer 710 usually relates to the number of input values of the neural network, and the number of nodes 731, 732 within the output layer 713 usually relates to the number of output values of the neural network.

In particular, a (real) number can be assigned as a value to every node 720, . . . , 732 of the neural network 700. Here, x⁽ⁿ⁾ᵢ denotes the value of the i-th node 720, . . . , 732 of the n-th layer 710, . . . , 713. The values of the nodes 720, . . . , 722 of the input layer 710 are equivalent to the input values of the neural network 700, the values of the nodes 731, 732 of the output layer 713 are equivalent to the output value of the neural network 700. Furthermore, each edge 740, . . . , 742 can comprise a weight being a real number, in particular, the weight is a real number within the interval [−1, 1] or within the interval [0, 1]. Here, w^{(m,n)}_{i,j} denotes the weight of the edge between the i-th node 720, . . . , 732 of the m-th layer 710, . . . , 713 and the j-th node 720, . . . , 732 of the n-th layer 710, . . . , 713. Furthermore, the abbreviation w⁽ⁿ⁾_{i,j} is defined for the weight w^{(n,n+1)}_{i,j}.

In particular, to calculate the output values of the neural network 700, the input values are propagated through the neural network. In particular, the values of the nodes 720, . . . , 732 of the (n+1)-th layer 710, . . . , 713 can be calculated based on the values of the nodes 720, . . . , 732 of the n-th layer 710, . . . , 713 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 710 are given by the input of the neural network 700, wherein values of the first hid-den layer 711 can be calculated based on the values of the input layer 710 of the neural network, wherein values of the second hidden layer 712 can be calculated based in the values of the first hidden layer 711, etc.

In order to set the values w^{(m,n)}_{i,j} for the edges, the neural network 700 has to be trained using training data. In particular, training data comprises training input data and training output data (denoted as tᵢ). For a training step, the neural network 700 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 700 (backpropagation algorithm). In particular, the weights are changed according to

$${w}^{{′ ( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 713, wherein f′ is the first derivative of the activation function, and t⁽ⁿ⁺¹⁾ⱼ is the comparison training value for the j-th node of the output layer 713.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernels are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

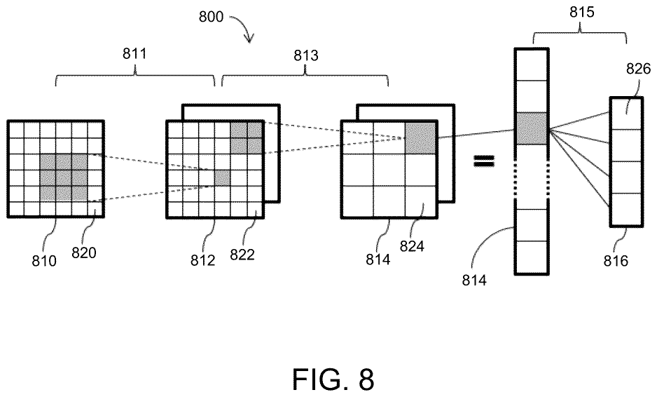

FIG. 8 shows an embodiment of a convolutional neural network 800 that may be used to implement one or more machine learning models described herein. In the displayed embodiment, the convolutional neural network 800 comprises an input node layer 810, a convolutional layer 811, a pooling layer 813, a fully connected layer 814 and an output node layer 816, as well as hidden node layers 812, 814. Alternatively, the convolutional neural network 800 can comprise several convolutional layers 811, several pooling layers 813 and several fully connected layers 815, as well as other types of layers. The order of the layers can be chosen arbitrarily, usually fully connected layers 815 are used as the last layers before the output layer 816.

In particular, within a convolutional neural network 800 nodes 820, 822, 824 of a node layer 810, 812, 814 can be considered to be arranged as a d-dimensional matrix or as a d-dimensional image. In particular, in the two-dimensional case the value of the node 820, 822, 824 indexed with i and j in the n-th node layer 810, 812, 814 can be denoted as x(n)[i, j]. However, the arrangement of the nodes 820, 822, 824 of one node layer 810, 812, 814 does not have an effect on the calculations executed within the convolutional neural network 800 as such, since these are given solely by the structure and the weights of the edges.

A convolutional layer 811 is a connection layer between an anterior node layer 810 (with node values x(n−1)) and a posterior node layer 812 (with node values x(n)). In particular, a convolutional layer 811 is characterized by the structure and the weights of the incoming edges forming a convolution operation based on a certain number of kernels. In particular, the structure and the weights of the edges of the convolutional layer 811 are chosen such that the values x(n) of the nodes 822 of the posterior node layer 812 are calculated as a convolution x(n)=K*x(n−1) based on the values x(n−1) of the nodes 820 anterior node layer 810, where the convolution * is defined in the two-dimensional case as

$${x}_{k}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 820, 822 (e.g., a 3×3 matrix, or a 5×5 matrix). In particular, this implies that the weights of the edges in the convolution layer 811 are not independent, but chosen such that they produce said convolution equation. In particular, for a kernel being a 3×3 matrix, there are only 9 independent weights (each entry of the kernel matrix corresponding to one independent weight), irrespectively of the number of nodes 820, 822 in the anterior node layer 810 and the posterior node layer 812.

In general, convolutional neural networks 800 use node layers 810, 812, 814 with a plurality of channels, in particular, due to the use of a plurality of kernels in convolutional layers 811. In those cases, the node layers can be considered as (d+1)-dimensional matrices (the first dimension indexing the channels). The action of a convolutional layer 811 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾^{ₐ} corresponds to the a-th channel of the anterior node layer 810, x(n) corresponds to the b-th channel of the posterior node layer 812 and K_{a,b} corresponds to one of the kernels. If a convolutional layer 811 acts on an anterior node layer 810 with A channels and outputs a posterior node layer 812 with B channels, there are A·B independent d-dimensional kernels K_{a,b}.

In general, in convolutional neural networks 800 activation functions are used. In this embodiment ReLU (acronym for “Rectified Linear Units”) is used, with R(z)=max (0, z), so that the action of the convolutional layer 811 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 810 comprises 36 nodes 820, arranged as a two-dimensional 6×6 matrix. The first hidden node layer 812 comprises 72 nodes 822, arranged as two two-dimensional 6×6 matrices, each of the two matrices being the result of a convolution of the values of the input layer with a 3×3 kernel within the convolutional layer 811. Equivalently, the nodes 822 of the first hidden node layer 812 can be interpreted as arranged as a three-dimensional 2×6×6 matrix, wherein the first dimension correspond to the channel dimension.

The advantage of using convolutional layers 811 is that spatially local correlation of the input data can exploited by enforcing a local connectivity pattern between nodes of adjacent layers, in particular by each node being connected to only a small region of the nodes of the preceding layer.

A pooling layer 813 is a connection layer between an anterior node layer 812 (with node values x(n−1)) and a posterior node layer 814 (with node values x(n)). In particular, a pooling layer 813 can be characterized by the structure and the weights of the edges and the activation function forming a pooling operation based on a non-linear pooling function f. For example, in the two-dimensional case the values x(n) of the nodes 824 of the posterior node layer 814 can be calculated based on the values x(n−1) of the nodes 822 of the anterior node layer 812 as

$${x}^{{( n )}_{b}} [ i , j ] = f ( {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 813 the number of nodes 822, 824 can be reduced, by re-placing a number d1·d2 of neighboring nodes 822 in the anterior node layer 812 with a single node 822 in the posterior node layer 814 being calculated as a function of the values of said number of neighboring nodes. In particular, the pooling function f can be the max-function, the average or the L2-Norm. In particular, for a pooling layer 813 the weights of the incoming edges are fixed and are not modified by training.

The advantage of using a pooling layer 813 is that the number of nodes 822, 824 and the number of parameters is reduced. This leads to the amount of computation in the network being reduced and to a control of overfitting.

In the displayed embodiment, the pooling layer 813 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 800 are fully connected layers 815. A fully connected layer 815 is a connection layer between an anterior node layer 814 and a posterior node layer 816. A fully connected layer 813 can be characterized by the fact that a majority, in particular, all edges between nodes 814 of the anterior node layer 814 and the nodes 816 of the posterior node layer are present, and wherein the weight of each of these edges can be adjusted individually.

In this embodiment, the nodes 824 of the anterior node layer 814 of the fully connected layer 815 are displayed both as two-dimensional matrices, and additionally as non-related nodes (indicated as a line of nodes, wherein the number of nodes was reduced for a better presentability). This operation is also denoted as “flattening”. In this embodiment, the number of nodes 826 in the posterior node layer 816 of the fully connected layer 815 smaller than the number of nodes 824 in the anterior node layer 814. Alternatively, the number of nodes 826 can be equal or larger.

Furthermore, in this embodiment the Softmax activation function is used within the fully connected layer 815. By applying the Softmax function, the sum the values of all nodes 826 of the output layer 816 is 1, and all values of all nodes 826 of the output layer 816 are real numbers between 0 and 1. In particular, if using the convolutional neural network 800 for categorizing input data, the values of the output layer 816 can be interpreted as the probability of the input data falling into one of the different categories.

In particular, convolutional neural networks 800 can be trained based on the backpropagation algorithm. For preventing overfitting, methods of regularization can be used, e.g., dropout of nodes 820, . . . , 824, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

A generative adversarial model (an acronym is GA model) comprises a generative function and a discriminative function, wherein the generative function creates synthetic data, and the discriminative function distinguishes between synthetic and real data. By training the generative function and/or the discriminative function on the one hand the generative function is configured to create synthetic data which is incorrectly classified by the discriminative function as real, on the other hand the discriminative function is configured to distinguish between real data and synthetic data generated by the generative function. In the notion of game theory, a generative adversarial model can be interpreted as a zero-sum game. The training of the generative function and/or of the discriminative function is based, in particular, on the minimization of a cost function.

By using a GA model, based on a set of training data synthetic data can be generated that has the same characteristics as the training data set. The training of the GA model can be based on data not being annotated (unsupervised learning), so that there is low effort in training a GA model.

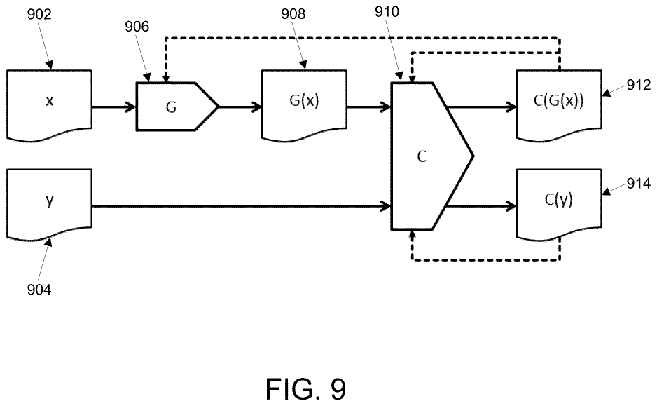

FIG. 9 shows a data flow diagram according to an embodiment for using a generative adversarial network for creating synthetic output data G(x) 908 based on input data x 902 that is indistinguishable from real output data y 904, in accordance with one or more embodiments. The synthetic output data G(x) 908 has the same structure as the real output data y 904, but its content is not derived from real world data.

The generative adversarial network comprises a generator function G 906 and a classifier function C 910 which are trained jointly. The task of the generator function G 906 is to provide realistic synthetic output data G(x) 908 based on input data x 902, and the task of the classifier function C 910 is to distinguish between real output data y 904 and synthetic output data G(x) 908. In particular, the output of the classifier function C 910 is a real number between 0 and 1 corresponding to the probability of the input value being real data, so that an ideal classifier function would calculate an output value of C(y) 914≈1 for real data y 904 and C(G(x)) 912≈0 for synthetic data G(x) 908.

Within the training process, parameters of the generator function G 906 are adapted so that the synthetic output data G(x) 908 has the same characteristics as real output data y 904, so that the classifier function C 910 cannot distinguish between real and synthetic data anymore. At the same time, parameters of the classifier function C 910 are adapted so that it distinguishes between real and synthetic data in the best possible way. Here, the training relies on pairs comprising input data x 902 and the corresponding real output data y 904. Within a single training step, the generator function G 906 is applied to the input data x 902 for generating synthetic output data G(x) 908. Furthermore, the classifier function C 910 is applied to the real output data y 904 for generating a first classification result C(y) 914. Additionally, the classifier function C 910 is applied to the synthetic output data G(x) 908 for generating a second classification result C(G(x)) 912.

Adapting the parameters of the generative function G 906 and the classifier function C 910 is based on minimizing a cost function by using the backpropagation algorithm, respectively. In this embodiment, the cost function K_{C} for the classifier function C 910 is K_{C}∝−BCE(C(y), 1)−BCE (C(G(x), 0), wherein BCE denotes the binary cross entropy defined as BCE(z, z′)=z′·log (z)+ (1−z′)·log (1−z). By using this cost function, both wrongly classifying real output data as synthetic (indicated by C(y)≈0) and wrongly classifying synthetic output data as real (indicated as C(G(x)) 912≈1) increases the cost function K_{C} to be minimized. Furthermore, the cost function K_{G} for the generator function G 906 is K_{G}∝−BCE(C(G(x), 1)=−log (C(G(x). By using this cost function, correctly classified synthetic output data (indicated as C(G(x)) 912≈0) leads to an increase of the cost function K_{G} to be minimized.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

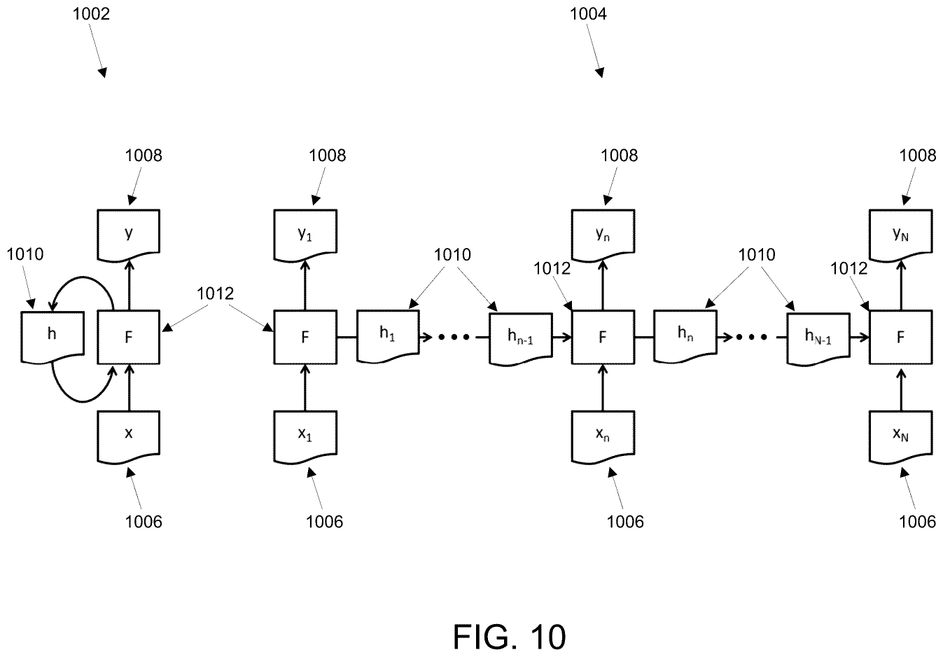

FIG. 10 shows the schematic structure of a recurrent machine learning model F, both in a recurrent representation 1002 and in an unfolded representation 1004, that may be used to implement one or more machine learning models described herein. The recurrent machine learning model takes as input several input datasets x, x₁, . . . , x_{N} 1006 and creates a corresponding set of output datasets y, y₁, . . . , y_{N} 1008. Furthermore, the output depends on a so-called hidden vector h, h₁, . . . , h_{N} 1010, which implicitly comprises information about input datasets previously used as input for the recurrent machine learning model F 1012. By using these hidden vectors h, h₁, . . . , h_{N} 1010, a sequentiality of the input datasets can be leveraged.

In a single step of the processing, the recurrent machine learning model F 1012 takes as input the hidden vector hₙ₋₁ created within the previous step and an input dataset xₙ. Within this step, the recurrent machine learning model F generates as output an updated hidden vector hₙ and an output dataset yₙ. In other words, one step of processing calculates (yₙ, hₙ)=F(xₙ, hₙ₋₁), or by splitting the recurrent machine learning model F 1012 into a part F(y) calculating the output data and F (h) calculating the hidden vector, one step of processing calculates yₙ=F^{(y)}(xₙ, hₙ₋₁) and hₙ=F^{(h)}(xₙ, hₙ₋₁). For the first processing step, h₀ can be chosen randomly or filled with all entries being zero. The parameters of the recurrent machine learning model F 1012 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F(h)(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-3. Certain steps or functions of the methods and workflows described herein, including one or more of the steps or functions of FIGS. 1-3, may be performed by a server or by another processor in a network-based cloud-computing system. Certain steps or functions of the methods and workflows described herein, including one or more of the steps of FIGS. 1-3, may be performed by a client computer in a network-based cloud computing system. The steps or functions of the methods and workflows described herein, including one or more of the steps of FIGS. 1-3, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-3, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 1102 that may be used to implement systems, apparatuses, and methods described herein is depicted in FIG. 11. Computer 1102 includes a processor 1104 operatively coupled to a data storage device 1112 and a memory 1110. Processor 1104 controls the overall operation of computer 1102 by executing computer program instructions that define such operations. The computer program instructions may be stored in data storage device 1112, or other computer readable medium, and loaded into memory 1110 when execution of the computer program instructions is desired. Thus, the method and workflow steps or functions of FIGS. 1-3 can be defined by the computer program instructions stored in memory 1110 and/or data storage device 1112 and controlled by processor 1104 executing the computer program instructions. For example, the computer program instructions can be implemented as computer executable code programmed by one skilled in the art to perform the method and workflow steps or functions of FIGS. 1-3. Accordingly, by executing the computer program instructions, the processor 1104 executes the method and workflow steps or functions of FIGS. 1-3. Computer 1102 may also include one or more network interfaces 1106 for communicating with other devices via a network. Computer 1102 may also include one or more input/output devices 1108 that enable user interaction with computer 1102 (e.g., display, keyboard, mouse, speakers, buttons, etc.).

Processor 1104 may include both general and special purpose microprocessors, and may be the sole processor or one of multiple processors of computer 1102. Processor 1104 may include one or more central processing units (CPUs), for example. Processor 1104, data storage device 1112, and/or memory 1110 may include, be supplemented by, or incorporated in, one or more application-specific integrated circuits (ASICs) and/or one or more field programmable gate arrays (FPGAs).

Data storage device 1112 and memory 1110 each include a tangible non-transitory computer readable storage medium. Data storage device 1112, and memory 1110, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1108 may include peripherals, such as a printer, scanner, display screen, etc. For example, input/output devices 1108 may include a display device such as a cathode ray tube (CRT) or liquid crystal display (LCD) monitor for displaying information to the user, a keyboard, and a pointing device such as a mouse or a trackball by which the user can provide input to computer 1102.

An image acquisition device 1114 can be connected to the computer 1102 to input image data (e.g., medical images) to the computer 1102. It is possible to implement the image acquisition device 1114 and the computer 1102 as one device. It is also possible that the image acquisition device 1114 and the computer 1102 communicate wirelessly through a network. In a possible embodiment, the computer 1102 can be located remotely with respect to the image acquisition device 1114.

Any or all of the systems, apparatuses, and methods discussed herein may be implemented using one or more computers such as computer 1102.

One skilled in the art will recognize that an implementation of an actual computer or computer system may have other structures and may contain other components as well, and that FIG. 11 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving 1) a first and a second medical image of an anatomical structure of a patient and 2) a mask of the anatomical structure in the second medical image; aligning the second medical image to the first medical image according to a deformation field; modifying the mask of the anatomical structure in the second medical image based on the deformation field to generate a pseudo-mask of the anatomical structure in the first medical image; and outputting the pseudo-mask of the anatomical structure in the first medical image.

Illustrative embodiment 2. The computer-implemented method of illustrative embodiment 1, further comprising: training a machine-learning-based task network for performing a medical imaging analysis task based on the first medical image and the pseudo-mask of the anatomical structure in the first medical image.

Illustrative embodiment 3. The computer-implemented method of illustrative embodiment 2, wherein training a machine-learning-based task network for performing a medical imaging analysis task based on the first medical image and the pseudo-mask of the anatomical structure in the first medical image comprises: iteratively repeating the receiving, the aligning, the modifying, and the outputting for a training dataset to generate a plurality of pseudo-masks, wherein the training dataset comprises first training images of the anatomical structure, second training images of the anatomical structure, and training masks of the anatomical structure in the second training images, and wherein the iteratively repeating is performed using one of the first training images as the first medical image, one of the second training images as the second medical image, and one of the training masks as the mask of the anatomical structure in the second medical image at each iteration; performing an initial training of the machine-learning-based task network for performing the medical imaging analysis task based on the second training images and the plurality of pseudo-masks; evaluating results of the medical imaging analysis task output by the initially trained machine-learning-based task network by comparing the results with the plurality of pseudo-masks to determine whether each of the plurality of pseudo-masks represent high confidence data or low confidence data; and retraining the machine-learning-based task network for performing the medical imaging analysis task based on the plurality of pseudo-masks that represent high confidence data, without using the plurality of pseudo-masks that represent low confidence data.

Illustrative embodiment 4. The computer-implemented method of illustrative embodiment 3, further comprising: adjusting the plurality of pseudo-masks that represent low confidence data.

Illustrative embodiment 5. The computer-implemented method of claim 2, wherein the medical imaging analysis task comprises segmentation of the anatomical structure from a medical image.

Illustrative embodiment 6. The computer-implemented method of any one of illustrative embodiments 2-5, wherein the medical imaging analysis task comprises detection of the anatomical structure from a medical image.

Illustrative embodiment 7. The computer-implemented method of any one of illustrative embodiments 1-6, wherein the first medical image and the second medical image are in different domains.

Illustrative embodiment 8. The computer-implemented method of any one of illustrative embodiments 1-7, wherein the first medical image comprises a q-Dixon image and the second medical image comprises an e-Dixon image.

Illustrative embodiment 9. The computer-implemented method of any one of illustrative embodiments 1-8, wherein aligning the second medical image to the first medical image according to a deformation field comprises: performing a rigid or non-rigid registration between the second medical image and the first medical image.

Illustrative embodiment 10. An apparatus comprising: means for receiving 1) a first and a second medical image of an anatomical structure of a patient and 2) a mask of the anatomical structure in the second medical image; means for aligning the second medical image to the first medical image according to a deformation field; means for modifying the mask of the anatomical structure in the second medical image based on the deformation field to generate a pseudo-mask of the anatomical structure in the first medical image; and means for outputting the pseudo-mask of the anatomical structure in the first medical image.

Illustrative embodiment 11. The apparatus of illustrative embodiment 10, further comprising: means for training a machine-learning-based task network for performing a medical imaging analysis task based on the first medical image and the pseudo-mask of the anatomical structure in the first medical image.

Illustrative embodiment 12. The apparatus of illustrative embodiment 11, wherein the means for training a machine-learning-based task network for performing a medical imaging analysis task based on the first medical image and the pseudo-mask of the anatomical structure in the first medical image comprises: means for iteratively repeating the means for receiving, the means for aligning, the means for modifying, and the means for outputting for a training dataset to generate a plurality of pseudo-masks, wherein the training dataset comprises first training images of the anatomical structure, second training images of the anatomical structure, and training masks of the anatomical structure in the second training images, and wherein the means for iteratively repeating is performed using one of the first training images as the first medical image, one of the second training images as the second medical image, and one of the training masks as the mask of the anatomical structure in the second medical image at each iteration; means for performing an initial training of the machine-learning-based task network for performing the medical imaging analysis task based on the second training images and the plurality of pseudo-masks; means for evaluating results of the medical imaging analysis task output by the initially trained machine-learning-based task network by comparing the results with the plurality of pseudo-masks to determine whether each of the plurality of pseudo-masks represent high confidence data or low confidence data; and means for retraining the machine-learning-based task network for performing the medical imaging analysis task based on the plurality of pseudo-masks that represent high confidence data, without using the plurality of pseudo-masks that represent low confidence data.

Illustrative embodiment 13. The apparatus of illustrative embodiment 12, further comprising: means for adjusting the plurality of pseudo-masks that represent low confidence data.

Illustrative embodiment 14. The apparatus of any one of illustrative embodiments 11-13, wherein the medical imaging analysis task comprises segmentation of the anatomical structure from a medical image.

Illustrative embodiment 15. The apparatus of any one of illustrative embodiments 11-14, wherein the medical imaging analysis task comprises detection of the anatomical structure from a medical image.

Illustrative embodiment 16. A non-transitory computer-readable storage medium comprising instructions which, when executed by a computer, cause the computer to carry out operations comprising: receiving 1) a first and a second medical image of an anatomical structure of a patient and 2) a mask of the anatomical structure in the second medical image; aligning the second medical image to the first medical image according to a deformation field; modifying the mask of the anatomical structure in the second medical image based on the deformation field to generate a pseudo-mask of the anatomical structure in the first medical image; and outputting the pseudo-mask of the anatomical structure in the first medical image.

Illustrative embodiment 17. The non-transitory computer-readable storage medium of illustrative embodiment 16, the operations further comprising: training a machine-learning-based task network for performing a medical imaging analysis task based on the first medical image and the pseudo-mask of the anatomical structure in the first medical image.

Illustrative embodiment 18. The non-transitory computer-readable storage medium of any one of illustrative embodiments 16-17, wherein the first medical image and the second medical image are in different domains.

Illustrative embodiment 19. The non-transitory computer-readable storage medium of any one of illustrative embodiments 16-18, wherein the first medical image comprises a q-Dixon image and the second medical image comprises an e-Dixon image.

Illustrative embodiment 20. The non-transitory computer-readable storage medium of any one of illustrative embodiments 16-19, wherein aligning the second medical image to the first medical image according to a deformation field comprises: performing a rigid or non-rigid registration between the second medical image and the first medical image.

1. A computer-implemented method comprising:
receiving 1) a first and a second medical image of an anatomical structure of a patient and 2) a mask of the anatomical structure in the second medical image;
aligning the second medical image to the first medical image according to a deformation field;
modifying the mask of the anatomical structure in the second medical image based on the deformation field to generate a pseudo-mask of the anatomical structure in the first medical image; and
outputting the pseudo-mask of the anatomical structure in the first medical image.

2. The computer-implemented method of claim 1, further comprising:
training a machine-learning-based task network for performing a medical imaging analysis task based on the first medical image and the pseudo-mask of the anatomical structure in the first medical image.

3. The computer-implemented method of claim 2, wherein training a machine-learning-based task network for performing a medical imaging analysis task based on the first medical image and the pseudo-mask of the anatomical structure in the first medical image comprises:
iteratively repeating the receiving, the aligning, the modifying, and the outputting for a training dataset to generate a plurality of pseudo-masks, wherein the training dataset comprises first training images of the anatomical structure, second training images of the anatomical structure, and training masks of the anatomical structure in the second training images, and wherein the iteratively repeating is performed using one of the first training images as the first medical image, one of the second training images as the second medical image, and one of the training masks as the mask of the anatomical structure in the second medical image at each iteration;
performing an initial training of the machine-learning-based task network for performing the medical imaging analysis task based on the second training images and the plurality of pseudo-masks;
evaluating results of the medical imaging analysis task output by the initially trained machine-learning-based task network by comparing the results with the plurality of pseudo-masks to determine whether each of the plurality of pseudo-masks represent high confidence data or low confidence data; and
retraining the machine-learning-based task network for performing the medical imaging analysis task based on the plurality of pseudo-masks that represent high confidence data, without using the plurality of pseudo-masks that represent low confidence data.

4. The computer-implemented method of claim 3, further comprising:
adjusting the plurality of pseudo-masks that represent low confidence data.

5. The computer-implemented method of claim 2, wherein the medical imaging analysis task comprises segmentation of the anatomical structure from a medical image.

6. The computer-implemented method of claim 2, wherein the medical imaging analysis task comprises detection of the anatomical structure from a medical image.

7. The computer-implemented method of claim 1, wherein the first medical image and the second medical image are in different domains.

8. The computer-implemented method of claim 1, wherein the first medical image comprises a q-Dixon image and the second medical image comprises an e-Dixon image.

9. The computer-implemented method of claim 1, wherein aligning the second medical image to the first medical image according to a deformation field comprises:
performing a rigid or non-rigid registration between the second medical image and the first medical image.

10. An apparatus comprising:
means for receiving 1) a first and a second medical image of an anatomical structure of a patient and 2) a mask of the anatomical structure in the second medical image;
means for aligning the second medical image to the first medical image according to a deformation field;
means for modifying the mask of the anatomical structure in the second medical image based on the deformation field to generate a pseudo-mask of the anatomical structure in the first medical image; and
means for outputting the pseudo-mask of the anatomical structure in the first medical image.

11. The apparatus of claim 10, further comprising:
means for training a machine-learning-based task network for performing a medical imaging analysis task based on the first medical image and the pseudo-mask of the anatomical structure in the first medical image.

12. The apparatus of claim 11, wherein the means for training a machine-learning-based task network for performing a medical imaging analysis task based on the first medical image and the pseudo-mask of the anatomical structure in the first medical image comprises:
means for iteratively repeating the means for receiving, the means for aligning, the means for modifying, and the means for outputting for a training dataset to generate a plurality of pseudo-masks, wherein the training dataset comprises first training images of the anatomical structure, second training images of the anatomical structure, and training masks of the anatomical structure in the second training images, and wherein the means for iteratively repeating is performed using one of the first training images as the first medical image, one of the second training images as the second medical image, and one of the training masks as the mask of the anatomical structure in the second medical image at each iteration;
means for performing an initial training of the machine-learning-based task network for performing the medical imaging analysis task based on the second training images and the plurality of pseudo-masks;
means for evaluating results of the medical imaging analysis task output by the initially trained machine-learning-based task network by comparing the results with the plurality of pseudo-masks to determine whether each of the plurality of pseudo-masks represent high confidence data or low confidence data; and
means for retraining the machine-learning-based task network for performing the medical imaging analysis task based on the plurality of pseudo-masks that represent high confidence data, without using the plurality of pseudo-masks that represent low confidence data.

13. The apparatus of claim 12, further comprising:
means for adjusting the plurality of pseudo-masks that represent low confidence data.

14. The apparatus of claim 11, wherein the medical imaging analysis task comprises segmentation of the anatomical structure from a medical image.

15. The apparatus of claim 11, wherein the medical imaging analysis task comprises detection of the anatomical structure from a medical image.

16. A non-transitory computer-readable storage medium comprising instructions which, when executed by a computer, cause the computer to carry out operations comprising:
receiving 1) a first and a second medical image of an anatomical structure of a patient and 2) a mask of the anatomical structure in the second medical image;
aligning the second medical image to the first medical image according to a deformation field;
modifying the mask of the anatomical structure in the second medical image based on the deformation field to generate a pseudo-mask of the anatomical structure in the first medical image; and
outputting the pseudo-mask of the anatomical structure in the first medical image.

17. The non-transitory computer-readable storage medium of claim 16, the operations further comprising:
training a machine-learning-based task network for performing a medical imaging analysis task based on the first medical image and the pseudo-mask of the anatomical structure in the first medical image.

18. The non-transitory computer-readable storage medium of claim 16, wherein the first medical image and the second medical image are in different domains.

19. The non-transitory computer-readable storage medium of claim 16, wherein the first medical image comprises a q-Dixon image and the second medical image comprises an e-Dixon image.

20. The non-transitory computer-readable storage medium of claim 16, wherein aligning the second medical image to the first medical image according to a deformation field comprises:
performing a rigid or non-rigid registration between the second medical image and the first medical image.
